(19) United States
(12) Patent Application Publication
(10) Pub. No.: US 2003/0101469 A1
(43) Pub. Date: May 29, 2003

(54) Cloned non-human mammals from contact inhibited donor cells

(76) Inventors: Kenneth Bondioli, Hamilton, NY (US); Jaqdeece Ramsoondar, Hamilton, NY (US); William Fodor, Madison, CT (US)

Correspondence Address: Jeffrey S. Steen, Carter, DeLuca, Farrell & Schmidt, LLP, Suite 225, 445 Broad Hollow Road, Melville, NY, 11747, US

(21) Appl. No.: 10/192,025

(22) Filed: Jul. 9, 2002

Related U.S. Application Data
(60) Provisional application No. 60/304,431, filed on Jul. 9, 2001.

(51) Int. Cl. A01K 67/027

(52) U.S. Cl. 800/17; 800/21

(57) ABSTRACT

Cloned non-human mammalian nuclear transfer units, embryos and animals are provided by introducing a contact-inhibited non-cycling mammalian donor cell in the G1 state or a nucleus thereof into an enucleated oocyte from a non-human mammal of the same species as the donor to form a nuclear transfer unit, implanting the nuclear transfer unit into the uterus of a surrogate mother, permitting the nuclear transfer unit to develop into the cloned non-human mammalian embryo and allowing the embryo to come to term.

### CROSS REFERENCE TO RELATED APPLICATION

This application claims priority to U.S. Provisional Serial No. 60/304,431 filed on Jul. 9, 2001, the contents of which are hereby incorporated by reference.

### BACKGROUND

1. Technical Field

The present disclosure relates to production of cloned non-human mammals.

2. Description of Related Art

Nuclear transfer is a procedure involving the replacement of the nucleus of one cell with that of another, permitting the creation of genetically identical individuals. Recent reports on the cloning of sheep and cattle through nuclear transfer, such as the birth of “Dolly” the cloned lamb (Wilmut et al. (1997) Nature 385:810-813), have focused much attention on this aspect of reproductive biology. Despite the ability to clone sheep and cattle using nuclear transfer, a need exists for methods that will improve the probability of success of developing mammalian oocytes to produce embryos, fetuses, and eventually, infant and adult animals for agricultural, biomedical, and basic research purposes.

Cloning by nuclear transfer in domestic species was first accomplished by the fusion of sheep embryonic cells to enucleated unfertilized oocytes (Willadsen S M. 1986. Nuclear transplantation in sheep embryos. Nature 320:63-65; Smith L C and Wilmut I. 1989. Influence of nuclear and cytoplasmic activity on the development in vivo of sheep embryos after nuclear transplantation. Biol Reprod 40:1027-1035). These procedures were later successfully applied to cattle (Prather R S, Barnes F L, Sims M M, Robl J M, Eyestone W H, First N L. 1987. Nuclear transplantation in the bovine embryo: assessment of donor nuclei and recipient oocyte. Biol Reprod 37:859-866; Willadsen S M. 1989. Cloning of sheep and cow embryos. Genome 31:956-962; Bondioli K R, Westhusin M E, Looney C R. 1990. Production of identical bovine offspring by nuclear transfer. Theriogenology 33:165-174; Barnes F, Endebrock M, Looney C, Powell R, Westhusin M, Bondioli K. 1993. Embryo cloning in cattle: the use of in vitro matured oocytes. J Reprod Fertil. 97:317-320; Stice S L, Keefer C L, Matthews L. 1994. Bovine nuclear transfer embryos: Oocyte activation prior to blastomere fusion. Mol Reprod Dev 38:61-68), goats (Yong Z and Yuqiang L. 1998. Nuclear-cytoplasmic interaction and development of goat embryos reconstructed by nuclear transplantation: production of goats by serially cloning embryos. Biol Reprod 58:266-269) and pigs (Prather R S, Sims M M, First N L. 1989. Nuclear transplantation in early pig embryos. Biol Reprod 41:414-418). Nuclear transfer with early embryonic cells did not have much application for the production of transgenic animals since the donor cells were not grown in culture and not readily available for genetic manipulation. The reports by Campbell and Wilmut (Campbell K H, McWhir J, Ritchie W A, Wilmut I. 1996. Sheep cloned by nuclear transfer from a cultured cell line. Nature 380:64-66; Wilmut I, Schnieke A E, McWhir J, Kind A J, Campbell K H. 1997. Viable offspring derived from fetal and adult mammalian cells. Nature 385:810-813) that live sheep were born after nuclear transfer with donor nuclei from cultured cells including somatic cells of an adult animal, created a new opportunity for the production of transgenic animals including those with site specific genetic alterations (Schnieke A E, Kind A J, Ritchie W A, Mycock K, Scott A R, Ritchie M, Wilmut I, Colman A, Campbell K H. 1997. Human factor IX transgenic sheep produced by transfer of nuclei from transfected fetal fibroblasts. Science 278:2130-2133; McCreath K J, Howcroft J, Campbell K H, Colman A, Schnieke A E, Kind A J. 2000. Production of gene-targeted sheep by nuclear transfer from cultured somatic cells. Nature 405:1066-1069). The ability to produce pigs containing site specific genetic alterations induced by homologous recombination would have great value in the development of these animals for human therapeutic applications.

Live births of cloned pigs produced by somatic cell nuclear transfer have been reported by at least three independent groups (Betthauser J, Forsberg E, Augenstein M, Childs L, Eilertsen K, Enos J, Forsythe T, Golueke P, Jurgella G, Koppang R, Lesmeister T, Mallon K, Mell G, Misica P, Pace M, Pfister-Genskow M, Strelchenko N, Voelker G, Watt S, Thompson S, Bishop M. 2000. Production of cloned pigs from in vitro systems. Nat Biotechnol 18:1055-1059; Polejaeva I A, Chen S H, Vaught T D, Page R L, Mullins J, Ball S, Dai Y, Boone J, Walker S, Ayares D L, Colman A, Campbell K H. 2000. Cloned pigs produced by nuclear transfer from adult somatic cells. Nature 407:86-90; Onishi A, Iwamoto M, Akita T, Mikawa S, Takeda K, Awata T, Hanada H, Perry A C. 2000. Pig cloning by microinjection of fetal fibroblast nuclei. Science 289:1188-1190). In the report by Onishi et al., supra, early passage (three population doublings) fetal fibroblast nuclei were injected into the cytoplasm of enucleated oocytes (Onishi et al., 2000). Polejaeva et al. (2000) produced cloned pigs utilizing granulosa cells from a primary culture (time in culture not specified) in a double nuclear transfer procedure. In a third report, fetal fibroblasts at passage 0 or 2 were fused to enucleated oocytes and resulted in live births (Betthauser et al., 2000). U.S. Pat. Nos. 5,945,577, 6,215,041 and 6,235,969 describe a method of nuclear transfer involving the transplantation of proliferating or non-quiescent differentiated donor cells or cell nuclei into enucleated oocytes of the same species as the donor cell. U.S. Pat. No. 6,147,276 describes quiescent cell populations for nuclear transfer. Quiescent donor cells described therein are those that have been caused to exit the G1 phase and arrest at the G0 state. For additional disclosures directed to cloning, see also, U.S. Pat. Nos. 6,395,958, 6,258,998, 6,107,543, and 6,011,197.

### SUMMARY

A method of producing a cloned non-human mammalian embryo by nuclear transfer is provided which includes introducing a contact-inhibited non-cycling mammalian donor cell in the G1 state or a nucleus thereof into an enucleated oocyte from a non-human mammal of the same species as the donor to form a nuclear transfer unit, implanting the nuclear transfer unit into the uterus of a surrogate mother and permitting the nuclear transfer unit to develop into the cloned non-human mammalian embryo. In one embodiment, the non-human mammal may be an ungulate. The donor cell or donor cell nucleus may, for example, be an epithelial cell, neural cell, epidermal cell, keratinocyte, hematopoietic cell, melanocyte, chondrocyte, B-lymphocyte, T-lymphocyte, erythrocyte, macrophage, monocyte, fibroblast, muscle cell, or nuclei isolated therefrom. The non-human mammalian embryo can be allowed to mature and become a neonate.

A method of producing a cloned non-human mammalian nuclear transfer unit by nuclear transfer is also provided which includes introducing a contact-inhibited non-cycling mammalian donor cell in the G1 state or a nucleus thereof into an enucleated oocyte from a non-human mammal of the same species as the donor to form a nuclear transfer unit. In one embodiment, the species is an ungulate. The donor cell or donor cell nucleus may, for example, be an epithelial cell, neural cell, epidermal cell, keratinocyte, hematopoictic cell, melanocyte, chondrocyte, B-lymphocyte, T-lymphocyte, erythrocyte, macrophage, monocyte, fibroblast, muscle cell, or nuclei isolated therefrom. The nuclear transfer unit can be implanted into a surrogate mother and allowed to mature into an embryo.

In one embodiment, cloned pigs were produced from cultured skin fibroblasts derived from a H-transferase transgenic boar. One 90 day fetus and two healthy piglets resulted from nuclear transfer by fusion of cultured fibroblasts with enucleated oocytes. The cells used were subjected to an extensive culture time, freezing and thawing and clonal expansion from single cells prior to nuclear transfer. PCR and FACS analysis determined that the cloned offspring contained and expressed the H-transferase transgene. Micro-satellite analysis confirmed that the clones were genetically identical to the boar. The cell culture and nuclear transfer procedures described here are useful for applications requiring multiple genetic manipulations in the same animal.

### BRIEF DESCRIPTION OF THE DRAWINGS

FIG. 1 depicts cloned pigs 39-1 and 39-2 at two months of age.

FIG. 2 depicts a gel showing PCR amplification of the HT transgene. Lane 1, no NA control; lane 2, clone 39-1; lane 3, clone 39-2; lane 4, Recipient 2077; lanes 5 and 6, progeny of 1-12 by natural mating; lane 7, 1-12 and lane 8, FC 10.

FIG. 3 depicts graphs showing FACS analysis of fibroblasts from the control recipient, the boar from which donor cells were isolated and all three clones. Black dashed line=unstained cells. Gray dashed line=cells stained with UEAI (fucose specific lectin). Black dotted line=cells stained with IB4 (gal specific lectin).

### DETAILED DESCRIPTION OF PREFERRED EMBODIMENTS

The present disclosure provides improved procedures for cloning non-human mammals by nuclear transfer (also known as nuclear transplantation). Cloned non-human animals are produced by obtaining a contact-inhibited non-cycling donor cell in the G1 state or a nucleus thereof and effecting nuclear transfer into an enucleated oocyte obtained from a non-human mammal of the same species. The use of contact-inhibited non-cycling cells in the G1 state for the production of cloned non-human mammals provides an increased likelihood of success due to the fact that such donor cells are more stable compared to donor cells which are proliferating or non-quiescent. Such actively dividing cells go through numerous cycles in which genetic information within the cells is modified. Accordingly, use of such donor cells presents a non-uniform population which may lead to inconsistent results and potential abnormalities. On the other hand, donor cells which have been forced into quiescence via serum starvation were reported to have undergone extensive DNA fragmentation after three days of serum starvation (Kues W A, Anger M, Carnwath J W, Paul D, Motlik J, Niemann H. 2000. Cell cycle synchronization of porcine fetal fibroblasts: effects of serum deprivation and reversible cell cycle inhibitors. Biol Reprod 62:412-419). It has been reported that use of donor cells in the G0 state does not necessarily provide an increased likelihood of success in full term development. See, Boquest et al., Biol. Reprod., 60, 1013-1019 (1999). In addition, cells in the G0 state may become sinescent and irreversibly leave the cell division cycle which may interfere with the ability to produce a viable clone.

Non-cycling contact-inhibited donor cells in the G1 state are obtained by growing colonies of potential donor cells to confluence. After reaching confluence, a suitable period is allowed to pass, e.g., greater that 4 days, such that the cells are stable just prior to chromosome duplication (G1 phase). Those skilled in the art will recognize entry into the G1 phase and can suitably determine, depending on the potential donor cell type, the appropriate time frame for development of non-cycling G1 state contact inhibition. Such a determination may be carried out, for example, by using flow cytometric cell cycle analysis to measure cellular DNA and protein content. See, e.g., Boquest et al., supra, incorporated herein by reference. Thus, a cloned non-human mammalian embryo is produced by nuclear transfer by introducing a contact-inhibited non-cycling mammalian donor cell in the G1 state or a nucleus thereof into an enucleated oocyte from a non-human mammal of the same species as the donor to form a nuclear transfer unit, implanting the nuclear transfer unit into the uterus of a surrogate mother and permitting the nuclear transfer unit to develop into the cloned non-human mammalian embryo.

Nuclear transfer techniques or nuclear transplantation techniques are known in the literature and are described in many of the references cited above. See also, Campbell et al, Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci., USA, 90:6143-6147 (1993); WO 94/26884; WO 94/24274, and WO 90/03432, which are incorporated by reference in their entirety herein. Also, U.S. Pat. Nos. 4,944,384 and 5,057,420 describe procedures for bovine nuclear transplantation.

Potential donor cells may be obtained by well known methods. Mammalian cells useful herein include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells may be obtained from any cell or organ of the body. This includes all somatic or germ cells. Fibroblast cells are a preferred cell type because they can be obtained from developing fetuses and adult animals in large quantities.

Suitable mammalian sources for oocytes include sheep, cows, pigs, horses, rabbits, guinea pigs, mice, hamsters, rats, primates, etc. Preferably, the oocytes will be obtained from ungulates, and most preferably pigs. Methods for isolation of oocytes are well known in the art. Oocytes are typically isolated from the ovaries or reproductive tract of a mammal, e.g., pig, cow, etc. A readily available source of swine or bovine oocytes is slaughterhouse materials.

After a fixed time maturation period, which may range, e.g., from about 10 to 40 hours, the isolated oocytes will be enucleated. Prior to enucleation the oocytes may be placed in HECM containing 1 milligram per milliliter of hyaluronidase prior to removal of cumulus cells. This may be accomplished by repeated pipetting through very fine bore pipettes or by vortexing briefly. The stripped oocytes may then be screened for polar bodies, and selected metaphase II oocytes, as determined by the presence of polar bodies, may then be used for nuclear transfer. Enucleation may be effected by known methods, such as described in U.S. Pat. No. 4,994,384, which is incorporated by reference herein. For example, metaphase II oocytes are either placed in HECM, optionally containing 7.5 micrograms per milliliter cytochalasin B, for immediate enucleation, or may be placed in a suitable medium, for example an embryo culture medium such as CR1aa, plus 10% estrus cow serum, and then enucleated later, preferably not more than 24 hours later, and more preferably 16-18 hours later. Enucleation may be accomplished microsurgically using a micropipette to remove the polar body and the adjacent cytoplasm. The oocytes may then be screened to identify those of which have been successfully enucleated. This screening may be accomplished by staining the oocytes with 1 microgram per milliliter 33342 Hoechst dye in HECM, and then viewing the oocytes under ultraviolet irradiation for less than 10 seconds. The oocytes that have been successfully enucleated can then be placed in a suitable culture medium, e.g., CR1aa plus 10% serum.

The contact inhibited non-cycling donor cell in the G1 state or nucleus thereof and the enucleated oocyte are used to produce nuclear transfer (NT) units according to methods known in the art. Introduction of a membrane-bound nucleus from a donor mammalian cell into an enucleated recipient mammalian oocyte to form an oocyte containing the donor nucleus can be performed by fusing together the membrane of the membrane-bound nucleus from the donor mammalian cell with the membrane of the enucleated recipient mammalian oocyte to form an oocyte containing the nucleus from the donor mammalian cell. Alternatively, such introduction can be performed by microinjecting the membrane-bounded nucleus from the mammalian donor cell into the enucleated recipient mammalian oocyte to form an oocyte containing the nucleus from the donor mammalian cell. As those of ordinary skill in the art are aware, further alternative means exist for introducing donor nuclear material into a recipient oocyte. For example, and not by way of limitation, one can introduce a donor cell (or nucleus) into the space under the zona pellucida or into the perivitelline space of the enucleated, recipient oocyte, and then subsequently carry out membrane fusion to produce an oocyte containing within its cytoplasm the donor nucleus. For example, the cells may be fused by electrofusion. Electrofusion is accomplished by providing a pulse of electricity that is sufficient to cause a transient breakdown of the plasma membrane. This breakdown of the plasma membrane is very short because the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384. A variety of electrofusion media can be used including e.g., sucrose, mannitol, sorbitol and phosphate buffered solution. Fusion can also be accomplished using Sendai virus as a fusogenic agent Al1 means of introducing donor nuclear material into an enucleated recipient mammalian oocyte known to those of ordinary skill in the art are useful in the methods disclosed herein.

After fusion, the resultant fused NT units may then be placed in a suitable medium until activation, e.g., CR1aa medium. Typically activation will be effected shortly thereafter, typically less than 24 hours later, and preferably about 4-9 hours later. The NT unit may be activated by known methods. Such methods include, e.g., culturing the NT unit at sub-physiological temperature, in essence by applying a cold, or actually cool temperature shock to the NT unit. This may be done by culturing the NT unit at room temperature, which is cold relative to the physiological temperature conditions to which embryos are normally exposed. Alternatively, activation may be achieved by application of known activation agents. For example, penetration of oocytes by sperm during fertilization has been shown to activate prefusion oocytes to yield greater numbers of viable pregnancies and multiple genetically identical calves after nuclear transfer. Also, treatments such as electrical and chemical shock may be used to activate NT embryos after fusion. Examples of oocyte activation methods are disclosed in U.S. Pat. No. 5,496,720, herein incorporated by reference in its entirety. In addition, activation may be effected by simultaneously or sequentially (i) increasing levels of divalent cations in the oocyte, and (ii) reducing phosphorylation of cellular proteins in the oocyte. This may be accomplished by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators. Phosphorylation may be reduced by known methods, e.g., by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, e.g., phosphatase 2A and phosphatase 2B.

The activated NT units may then be cultured in a suitable in vitro culture medium. Culture media suitable for culturing and maturation of embryos are well known in the art. Examples of known media, which may be used for embryo culture and maintenance, include Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media. A common media used for the collection and maturation of oocytes is TCM-199, and 1 to 20% serum supplement including fetal calf serum, newborn serum, estrual cow serum, lamb serum or steer serum. In one example, a maintenance medium may include TCM-199 with Earl salts, 10% fetal calf serum, 0.2 mM Na pyruvate and 50 .mu.g/ml gentamicin sulphate. Any of the above may also involve co-culture with a variety of cell types such as granulosa cells, oviduct cells, BRL cells and uterine cells and STO cells. Another maintenance medium is described in U.S. Pat. No. 5,096,822, incorporated herein by reference. This embryo medium, named CR1, contains nutritional substances necessary to support an embryo. CR1 contains hemicalcium L-lactate in amounts ranging from 1.0 mM to 10 mM. Hemicalcium L-lactate is L-lactate with a hemicalcium salt incorporated thereon. Hemicalcium L-lactate is significant in that a single component satisfies two major requirements in the culture medium: (i) the calcium requirement necessary for compaction and cytoskeleton arrangement; and (ii) the lactate requirement necessary for metabolism and electron transport. Hemicalcium L-lactate also serves as valuable mineral and energy source for the medium necessary for viability of the embryos. Examples of the main components in CR1 medium include hemicalcium L-lactate, sodium chloride, potassium chloride, sodium bicarbonate and a minor amount of fatty-acid free bovine serum albumin (Sigma A-6003). Additionally, a defined quantity of essential and non-essential amino acids may be added to the medium. CR1 with amino acids is known by the abbreviation “CR1aa.”

Afterward, the cultured NT unit or units are preferably washed and then placed in a suitable media, e.g., CR1aa medium containing 10% FCS and 6 mg/ml contained in well plates which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells. Preparation of a suitable feeder layer is well within the skill of the ordinary artisan. The NT units are cultured on the feeder layer until the NT units reach a size suitable for transferring to a recipient female. Advantageously, these NT units will be cultured until at least about 2 to 400 cells. The culturing will be effected under suitable conditions, i.e., about 38.5° C. and 5% CO₂, with the culture medium changed in order to optimize growth typically about every 2-5 days, preferably about every 3 days. The methods for embryo transfer and recipient animal management are standard procedures used in the embryo transfer industry.

It is contemplated that adult mammals with desired genotypes can be produced according to the present disclosure. Multiplication of adult ungulates with proven genetic superiority or other desirable traits is particularly useful, including transgenic or genetically engineered animals, and chimeric animals. Furthermore, cell and tissues from the NT fetus, including transgenic and/or chimeric fetuses, can be used in cell, tissue and organ transplantation for the treatment of numerous diseases. For example, donor cells derived from a non-human mammal such as a pig which carries the H-transferase gene can be utilized to produce cloned pigs which all carry the gene. Organs from such cloned pigs may be utilized in cross-species xenotransplantation.

The following examples are included for purposes of illustration and should not be considered as limiting the scope of the present disclosure.

### EXAMPLE 1

Manipulation and Culture Media

Oocyte recovery and all manipulations were conducted in Beltsville Embryo Culture Medium (BECM)(Purse1 VG and Wall R J. 1996. Effects of transferred ova per recipient and dual use of donors as recipients on production of transgenic swine. Theriogenology 46:201-209). Following fusion of enucleated oocytes and donor fibroblasts, nuclear transfer embryos were cultured in NCSU-23 medium (Petters R M and Wells K D. 1993. Culture of pig embryos. J Reprod Fertil Suppl 48:61-73).

Superovulation of Gilts and Collection of Oocytes.

Unfertilized oocytes were recovered from either prepubertal or cycling gilts. In the case of cycling gilts, estrus was synchronized by oral administration of 18 mg altrenogest (Regu-Mate, Hoechst) daily. Altrenogest was given 5-9 days depending on the stage of the estrous cycle. Estrumate (250 μg, Bayer) was given on the last day of altrenogest treatment. In both cycling and prepubertal gilts superovulation was induced by a single im injection of 1,500 IU of PMSG (Diosynth) followed by 1,000 IU hCG 75 h later. In the case of cycling gilts PMSG was given 24 h after the last altrenogest treatment. Unfertilized oocytes were collected by retrograde flushing of the oviducts 48-50 h after the hCG injection.

Collection and Culture of Fibroblasts

Fibroblast cultures for nuclear transfer were established by outgrowth from minced earnotch samples of a four month old, H-transferase transgenic boar. The transgenic boar contains two single copy transgenes that encode the human H-transferase (HT) gene under the control of either the mouse h2kb promoter or the CMV promoter (Costa C, Zhao L, Burton W V, Bondioli K R, Williams B L, Hoagland T A, Ditullio P A, Ebert K M, Fodor W L. 1999. Expression of the human alphal, 2-fucosyltransferase in transgenic pigs modifies the cell surface carbohydrate phenotype and confers resistance to human serum-mediated cytolysis. FASEB J 13:1762-1773). Fibroblast culture was conducted in DMEM supplemented with 15% fetal calf serum (FCS) and 1% penicillin streptomycin (Gibco) at 38 C in a humidified atmosphere of 5% CO2 in air. Cells were frozen at passage three (25 days of culture) after isolation and stored for approximately two years for later use. Fibroblast cultures for flow cytometric analyses were also established by outgrowth from earnotch samples of the cloned pigs and the recipient. Skin from the cloned fetus and umbilical cord samples from the clones were also used to establish fibroblast cultures.

Transfection and Antibiotic Selection

Fibroblasts previously frozen at passage three were thawed and expanded by one passage prior to transfection by electroporation with a targeting construct containing a puromycin resistance gene. Cells were washed by centrifugation in electroporation medium and 800 μl of cell suspension (approximately 10⁷ cells) with 20 μg linearized plasmid were placed in a cuvette with a 4 mm gap. The electroporation medium consisted of 75% cytosalts (120 mM KCl; 0.15 mM CaCl₂; 10 mM K₂HPO₄, pH 7.6; 5 mM MgCl₂) (Van den Hoff M J B, Moorman A F M, Lamers W H. 1992. Electroporation in ‘intracellular’ buffer increases cell survival. Nucleic Acids Research 20:2902) and 25% Opti-MEM (Gibco). An electroporation pulse of 450V and 350 μF capacitance was delivered with a Bio Rad Gene Pulser. Electroporated cells were cultured in a 25 cm² tissue culture flask for 48 h to allow for expression of the puromycin resistance gene. Transfected cells were then selected in puromycin containg medium as follows. Cells were then trypsinized and plated in ten 100 mm tissue culture plates in culture medium containing 3 ug/ml puromycin. This concentration of puromycin was selected as the lowest dose which killed >95% of non transfected fibroblasts within three days (data not shown). Selection medium was changed at three day intervals until colonies of 100-200 cells appeared. Seventeen colonies were isolated and trypsinized with cloning rings and transferred to four well plates, one colony per well. Upon reaching near confluence 12 colonies were expanded to a single well of a 6 well plate and again allowed to reach near confluence. Ten of these cultures were trypsinized and split into two fractions, one to seed a single well of a 6 well plate and the other for DNA isolation for PCR analysis. Two clones C1 and C10 were selected for nuclear transfer experiments. Fibroblasts were trypsinized and replated into a single well of a 6 well plate several times during the nuclear transfer experiments. The cells used for nuclear transfer experiments were exposed to puromycin selection for a total of 30 days. Single cell clones were isolated and expanded. Nuclear transfer was performed with these cells after a total of 66 to 116 days in culture (Table I).

TABLE 1
Porcine cloning with cultured fibroblasts.

| | Nuclear | | | |
|---|---|---|---|---|
| | Donor | Days of | Number | |
| Recipient | Cell | Culture for | Embryos | |
| Number | Clone^{a} | Donor Cells | Transferred | Result |
| 2056 | C10 | 66 | 49 | Fetus Recovered^{b} |
| 2077 | C1 | 81 | 49 | Two Piglets Born^{c} |
| 2132 | C1 | 102 | 58 | Not Pregnant |
| 2159 | C1 | 110 | 37 | Not Pregnant |
| 2113 | C1 | 116 | 24 | Not Pregnant |

^{a}Skin fibroblasts transfected with a construct containing a puromycin resistance gene, selected with puromycin and expanded from a single cell.
^{b}Recipient was euthanized at 90 days of pregnancy for health reasons. One mummified and one apparently viable fetus were recovered.
^{c}Two normal health piglets delivered by caesarian section at 116 days of gestation.

Nuclear Transfer and Embryo Transfer

Two cloned cell lines were used as nuclear donors in five nuclear transfer experiments (Table I). In these experiments 229 oocytes were utilized, of which 217 survived the manipulation and were transferred. Fusion rates and cleavage rates were not determined and all embryos which survived manipulation were transferred. Donor cells were trypsinized immediately prior to nuclear transfer and placed in a drop of manipulation medium under oil. Manipulation medium consisted of BECM plus 7.5 μg/ml cytochalasin B. Unfertilized oocytes were enucleated 52-54 h after HCG injection by aspiration of the first polar body and a small amount of adjacent cytoplasm with a beveled pipette with an outer diameter of approximately 20 pm. A donor cell with a diameter of 12-15 μm was picked up with the same pipette and inserted into the space between the zona pellucida and the enucleated oocyte. Oocyte and cell combinations were equilibrated in Zimmerman's fusion medium (Zimmerman U and Vienken J. 1982. Electric field-induced cell to cell fusion. J Membr Biol 67:165-182; Wolfe B A and Kraemer D C. 1992. Methods in bovine nuclear transfer. Theriogenology 37:5-15) for 10 to 15 min before being placed between the electrodes of a fusion chamber. Fusion was induced by the delivery of a 6V AC alignment current followed by two DC pulses of 1.0 KV/cm and 60 μsec of duration. This process was repeated 20-30 min later and a second set of pulses delivered to aid in activation of the oocytes. Nuclear transfer embryos were cultured overnight in drops of NCSU 23 medium under oil at 39 C in a humidified atmosphere of 5% CO2 in air.

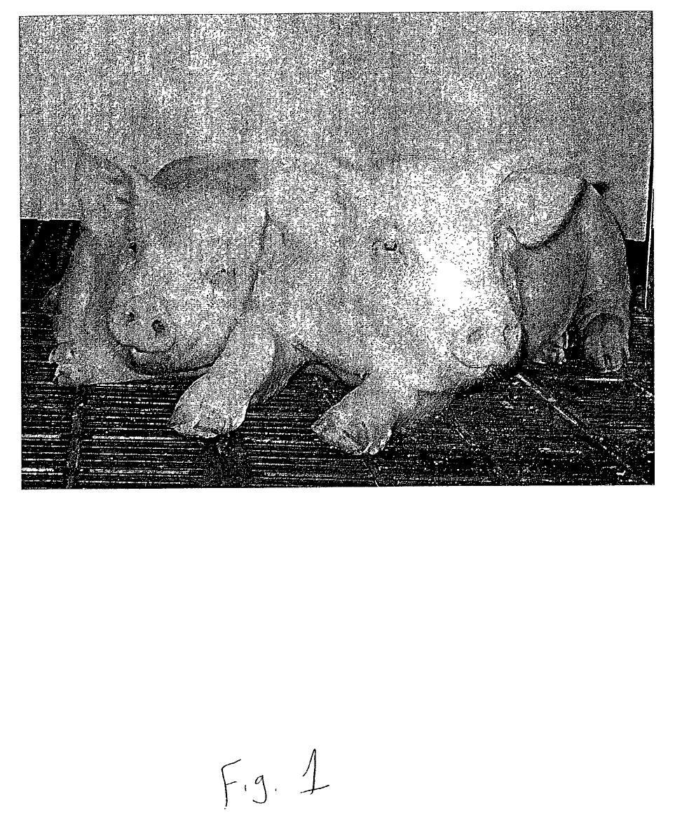

The following morning nuclear transfer embryos were transferred to the oviducts of recipients. Embryo transfer recipients were gilts programmed as oocyte donors 24 h later than the oocyte donors used for nuclear transfer. Embryo transfer was performed 48 h after hCG injection. Unfertilized oocytes were recovered from these animals immediately prior to embryo transfer. Two recipients became pregnant as a result of embryo transfer. One recipient was euthanized at approximately 90 days of gestation due to health problems unrelated to the embryo transfer. One mummified fetus and one apparently viable fetus were recovered. The viable fetus was designated FC10 and tissue was collected for isolation of genomic DNA and cell culture. Skin fibroblasts cultured from this tissue were used for expression analysis of the H transferase transgene. In addition cell aliquots were cryopreserved for later re-cloning. The second pregnancy produced two healthy piglets that were delivered by Caesarean section at 116 days of gestation. (FIG. 1).

PCR Analysis for the H-Transferase Transgene

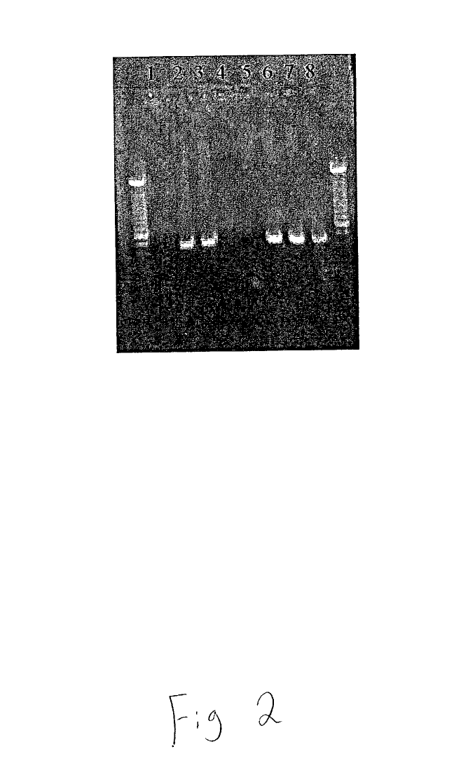

Genomic DNA from FC10 and the two clones (39-1 and 39-2) were analyzed by PCR for the presence of the HT transgene present in the transgenic boar (1-12) from which the nuclear donor cells were isolated. Genomic DNA was amplified with the PCR reaction utilizing forward (atgtcggaggagtacgcgg)(Seq. Id. No. 1) and reverse (agcagggcaaagtctttccac)(Seq. Id. No. 2) primers within the cDNA sequence of human H-transferase. The PCR reaction was carried out in 1×PCR buffer, 1.5 mM MgCl₂, 200 μM dNTPs and 20 pmoles of each primer in a final volume of 50 μi. Thermo cycling was performed in 0.5 ml tubes using an MJ Research PTC 100 thermal cycler (MJ Research, Waltharn, Mass.). Following an initial denaturing step of 3 min at 95 C the PCR reaction consisted of 35 cycles of 95 C, 55 C and 72 C each for 30 seconds. PCR products were run on a 1.2% agarose gel, stained with ethidum bromide and photographed. FC10, 39-1, 39-2, and the boar were positive for the HT transgene (FIG. 2). The recipient (2077) that gave birth to the clones was negative (FIG. 2). Genomic DNA samples were also analyzed for the presence of the puromycin resistance gene contained within the transfected construct. Neither FC10 nor the two live born clones were positive for the puromycin resistance gene. It is likely that some of the cells in the population used for nuclear transfer did not contain the puromycin gene and these cells resulted in the three clones. A similar result has been reported with neomycin selected porcine fetal fibroblasts (Betthauser et al., 2000). Skin fibroblast cultures were established from FC10, 39-1, 1-12 and 2077.

Flow Cytometric Analysis

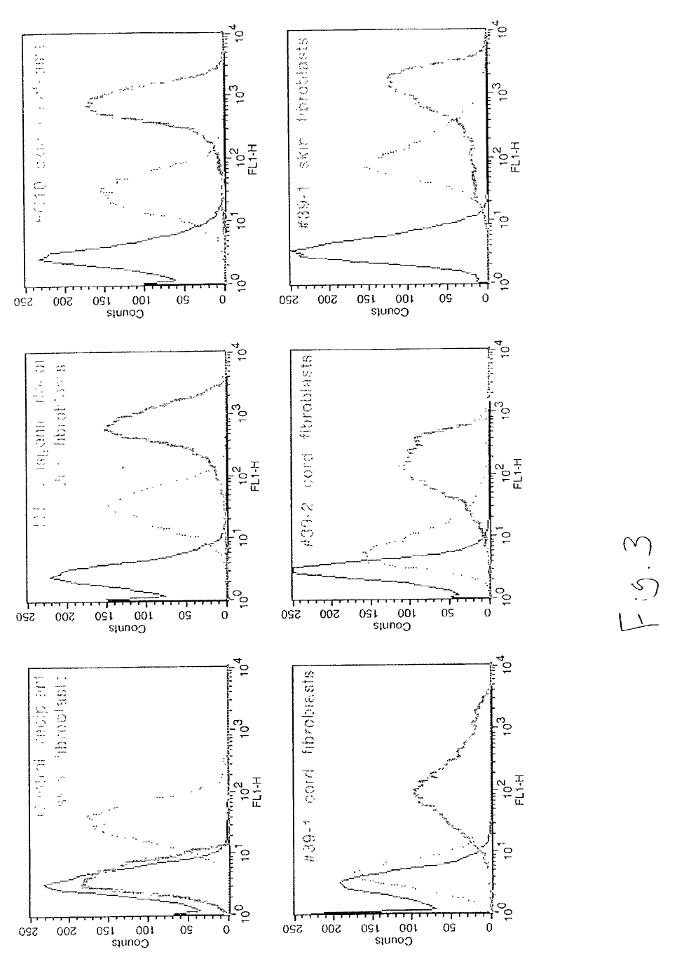

Umbilical cord fibroblasts were cultured from 39-1 and 39-2. These cells were analyzed for expression of the H-transferase transgene by flow cytometry (FIG. 3). Transgene expression was assessed by flow cytometry of primary cultured fibroblasts. Direct fluorescence of cell-surface carbohydrate epitopes was performed with fluorescein isothiocyanate (FITC)-conjugated lectins: IB4 lectin isolated from Griffonia simplicifolia (EY Laboratories, Inc. San Mateo, Calif.) detects Galα-1,3-Gal (Hayes C E and Goldstein I J. 1974. An α-D-galactosyl binding lectin from Bandeiraea simplicifolia seeds. J Biol Chem 249:1904-1914) and UEAI lectin isolated from Ulex europaeus (EY Laboratories) detects the H blood group (Matsumoto I and Osawa T. 1969. Purification and characterization of an antiH(0) phytohemagglutinin of Ulex europeus. Biochim Biophys Acta 194:189). Incubations were performed at 4 ″C for 30 minutes with 10 μg/ml of lectin in PBS plus 1% fetal bovine serum. Cell surface expression was then measured by flow cytometry on a Becton Dickinson FACSort. Fibroblasts from the recipient gave a staining pattern typical of normal porcine cells, high staining with the gal specific lectin (IB4) and virtually no staining with the anti-H blood group lectin UEA 1. Fibroblasts from the transgenic boar show a typical staining pattern for this transgene (Costa et al., 1999). Staining with UEA 1 is dramatically increased and IB 4 staining is lower than the control due to competition between gal transferase and H transferase for the same acceptor substrate molecule. Skin fibroblasts from FC10 and 39-1 gave almost identical patterns of H epitope expression to that of the transgenic boar. Fibroblasts isolated from the umbilical cords of 39-1 and 39-2 show a similar pattern of H epitope expression except there was slightly less staining with both lectins in this cell type. This difference is most likely due to the different cell source.

Micro Satellite Analysis

Micro satellite analysis with 16 markers was performed on genomic DNA from the two piglets born alive, the recovered fetus, the recipient and the boar from which the donor cells were taken (Table 2). Genomic DNA from the fetus FC10, the two live piglets, the boar from which donor cells were derived and the recipient which gave birth to the piglets were sent as coded samples to Celera-AgGEN (Davis, Calif.) for micro satellite analysis. The micro satellite analysis consisted of 16 polymorphic porcine loci consisting of different multimers of dinucleotide repeats. The PCR primer sequences for these loci are proprietary to Celera-AgGEN. One of the markers was indeterminate in all five samples. For the remaining 15 markers an identical genotype was obtained for 39-1, 39-2, FC10 and the boar 1-12. The recipient, 2077, had a different genotype for 13 of the 15 markers. The result of this analysis confirmed that the two piglets and the recovered fetus were genetically identical to each other and to the boar from which the cells were taken.

TABLE 2
Micro satellite analysis of cloned pigs.

| | | | Clone | Clone | |
|---|---|---|---|---|---|
| | Recipient | Boar 1-12 | 39-1 | 39-2 | Fetus FC10 |
| Loci | genotypes | Genotypes | Genotypes | Genotypes | Genotypes |
| S0059 | 150/158 | 150/150 | 150/150 | 150/150 | 150/150 |
| S0070 | 279/279 | 295/295 | 295/295 | 295/295 | 295/295 |
| S0122 | ND | ND | ND | ND | ND |
| S0226 | 192/192 | 180/180 | 180/180 | 180/180 | 180/180 |
| SW1430 | 160/160 | 160/166 | 160/166 | 160/166 | 160/166 |
| SW2160 | 175/183 | 187/187 | 187/187 | 187/187 | 187/187 |
| SW24 | 109/109 | 109/109 | 109/109 | 109/109 | 109/109 |
| SW2411 | 179/203 | 195/223 | 195/223 | 195/223 | 195/223 |
| SW2623 | 136/136 | 136/140 | 136/140 | 136/140 | 136/140 |
| SW72 | 110/110 | 114/114 | 114/114 | 114/114 | 114/114 |
| SW840 | 129/129 | 121/129 | 121/129 | 121/129 | 121/129 |
| SW893 | 109/123 | 117/117 | 117/117 | 117/117 | 117/117 |
| SW902 | 202/202 | 202/204 | 202/204 | 202/204 | 202/204 |
| SW936 | 95/109 | 95/109 | 95/109 | 95/109 | 95/109 |
| SWC27 | 139/163 | 163/167 | 163/167 | 163/167 | 163/167 |
| TNFB | 161/170 | 164/185 | 164/185 | 164/185 | 164/185 |

The data presented in Example 1 demonstrate that nuclear transfer in the pig can be accomplished with cells isolated from a 4 month old boar and that these cells can be cultured, cryopresetved and clonally expanded prior to use as nuclear donors.

The rate of development to term or near term reported here is similar to other published studies for porcine somatic cell nuclear transfer (Betthauser et al., 2000; Polejaeva et al., 2000; Onishi et al., 2000). The number of embryos transferred to each recipient in this study is comparable to that reported by Polejaeva et al. (2000), but considerably less than that reported by Betthauser et al. (2000). Intermediate efficiencies such as fusion rate and cleavage rate were not determined in these experiments. In other experiments fusion rates have been determined by staining the embryos the day after fusion. In these cases fusion rates are highly variable for different groups of oocytes but average approximately 50%. In these experiments and those reported earlier, the number of live animals born have been too small to accurately assess the efficiency of the techniques used. In contrast to other reports of pig somatic cell cloning (Polejaeva et al., 2000; Onishi et al., 2000) these cells were neither serum starved nor contact inhibited prior to use for nuclear transfer. Successful nuclear transfer with cells not subjected to serum starvation could be significant since it has been reported that porcine fibroblasts underwent extensive DNA fragmentation after three days of serum starvation (Kues et al., 2000).

The construct utilized in these experiments was designed to inactivate the α 1,3-galactosyltransferase (GT) gene and contained a puromycin resistance gene for selection. When genomic DNA from FC10 and the two live born clones were analyzed, this gene was not targeted nor was the puromycin resistance gene present. One explanation for the failure to detect the puromycin resistance gene after puromycin selection is the so called “by-stander effect”. Expression of an antibiotic resistance gene in some cells could confer antibiotic resistance to nearby cells either by direct contact between cells or secretion of the gene product into the medium. A similar result has been reported with neomycin selected porcine fetal fibroblasts (Betthauser et al., 2000).

There is much interest in the potential of xenotransplantation of genetically engineered pig cells and organs for human therapies (Auchincloss H J and Sachs D H. 1998. Xenogeneic transplantation. Annu Rev Immunol 16:433-470; Edge A S, Gosse M E, Dinsmore J. 1998. Xenogeneic cell therapy: current progress and future developments in porcine cell transplantation. Cell Transplant 7:525-539). A major barrier to xenogeneic transplantation is the phenomenon of Hyper Acute Rejection (HAR). The hyperacute response is triggered by the reaction of pre-formed antibodies reactive to the carbohydrate antigen, galactose α-1,3-galactose (αGal), an antigen absent from Old World primates because of the lack of a functional a 1,3-galactosyltransferase gene (Joziasse D H and Oriol R. 1999. Xenotransplantation: the importance of the Gal alpha 1,3Gal epitope in hyperacute vascular rejection. Biochim Biophys Acta 1455:403-418).

A major step towards the elimination of HAR in xenogeneic transplantation of porcine cells would be the elimination of this carbohydrate antigen from donor cells by the inactivation of the α 1,3-galactosyltransferase (GT) enzyme. The technology to inactivate genes with site-specific alterations by homologous recombination in cultured cells has been available for some time. Live mice containing such alterations are readily produced by gene targeting in embryonic stem cells (ES cells) (Thompson S, Clarke A R, Pow A M, Hooper M L, Melton D W. 1989. Germ line transmission and expression of a corrected HPRT gene produced by gene targeting in embryonic stem cells. Cell 56:313-321). So far ES cells which provide germline transmission of the ES cell genotype have not been developed for the pig. The discovery that nuclear transfer can be used to produce live animals with cultured cells including somatic cells provides an opportunity to produce live animals from porcine cells after successful homologous recombination.

If nuclear transfer in the pig is to be utilized to produce animals from cells in which precise gene targeting has been performed cells will have to be cultured for extended periods of time and survive clonal propagation. In some applications it will also be necessary to combine multiple genetic alterations in the same animal. For example if the GT enzyme is inactivated it will likely be beneficial to have an alternative enzyme such as H transferase. The combinations of genetic alterations in a single animal will be hastened if nuclear transfer can be accomplished with cells from previously characterized transgenic lines without the need of producing fetal cells from those lines. While porcine somatic cell nuclear transfer has been previously reported the results reported here are significant in several ways. These results expand the list of nuclear donor cell types that have generated live pigs. Non-fetal derived skin fibroblasts were used to produce the cloned pigs described here. The ability to utilize these cells will be beneficial in applications where multiple genetic alterations in the same animal will be required. As demonstrated here it will be possible to perform nuclear transfer with cells from a previously characterized transgenic line without the added step of first producing fetal cells from subsequent matings. It has also been reported that the efficiency of homologous recombination is higher if the homologous arms of the targeting construct are isogenic to the cells to be transfected (Deng C and Capecchi M R. 1992. Reexamination of gene targeting frequency as a function of the extent of homology between the targeting vector and the target locus. Mol Cell Biol 12:3365-3371). These results demonstrate the feasibility of obtaining isogenic DNA to build targeting constructs when inbred strains are not available. These results confirm those of Betthauser et al. (2000) that porcine nuclear transfer can be successful by fusion of intact cells with enucleated oocytes. It is clear that neither the complicated double nuclear transfer procedure reported by Polejaeva et al. (2000) nor intra cytoplasmic injection as reported by Onishi et al. (2000) are necessary to produce cloned pigs. As stated before, too few live pigs have been born to evaluate the efficiency of any method. Finally these results demonstrate that cells which have been extensively manipulated in culture can be used for nuclear transfer and can result in live births. These manipulations include extensive culture time, freezing and thawing and clonal expansion from single cells. Each of these steps will be of vital importance for somatic cell nuclear transfer to have utility in biomedical or agricultural applications.

### EXAMPLE 2

Skin fibroblast cells were isolated from a 28-day-old pig fetus and frozen at passage two. These cells were later thawed, made transgenic by transfection as described in Example 1 above and frozen for a second time at passage six. Following thawing for a second time the cells were cultured with continual passage at confluence for a period of approximately three months before nuclear transfer. Prior to nuclear transfer cells were allowed to come to confluence and were held at confluence for 14 days and a non-cycling G1 state achieved. Donor cells obtained in this manner were used for nuclear transfer and 68 nuclear transfer embryos were transferred as in Example 1 above to recipient number 2564. Three transgenic cloned piglets were born by cesarean section.

The above description sets forth preferred embodiments and examples. It should be understood that those skilled in the art will envision modifications of the embodiments and examples that, although not specifically stated herein, are still within the spirit and scope of any claims which may be appended hereto.

What is claimed is:

1. A method of producing a cloned non-human mammalian embryo by nuclear transfer comprising introducing a contact-inhibited non-cycling mammalian donor cell in the G1 state or a nucleus thereof into an enucleated oocyte from a non-human mammal of the same species as the donor to form a nuclear transfer unit, implanting the nuclear transfer unit into the uterus of a surrogate mother and permitting the nuclear transfer unit to develop into the cloned non-human mammalian embryo.

2. A method of producing a cloned non-human mammalian embryo by nuclear transfer according to claim 1 wherein the non-human mammalian embryo is an ungulate.

3. A method of producing a cloned non-human mammalian embryo by nuclear transfer according to claim 2 wherein the ungulate is a pig.

4. A method of producing a cloned non-human mammalian embryo by nuclear transfer according to claim 3 wherein the pig is an H-transferase transgenic pig.

5. A method of producing a cloned non-human mammalian embryo by nuclear transfer according to claim 1 wherein the donor cell or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, muscle cells, and nuclei isolated therefrom.

6. A method of producing a cloned non-human mammalian embryo by nuclear transfer according to claim 1 wherein the non-human mammalian embryo is allowed to mature and become a neonate.

7. A method of producing a cloned non-human mammalian nuclear transfer unit by nuclear transfer comprising introducing a contact-inhibited non-cycling mammalian donor cell in the G1 state or a nucleus thereof into an enucleated oocyte from a non-human mammal of the same species as the donor to form a nuclear transfer unit.

8. A method of producing a cloned non-human mammalian nuclear transfer unit by nuclear transfer according to claim 7 wherein the species is an ungulate.

9. A method of producing a cloned non-human mammalian nuclear transfer unit by nuclear transfer according to claim 8 wherein the ungulate is a pig.

10. A method of producing a cloned non-human mammal by nuclear transfer according to claim 9 wherein the pig is an H-transferase transgenic pig.

11. A method of producing a cloned non-human mammalian nuclear transfer unit by nuclear transfer according to claim 7 wherein the donor cell or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, muscle cells, and nuclei isolated therefrom.

12. A method of producing a cloned non-human mammalian nuclear transfer unit by nuclear transfer according to claim 7 wherein the nuclear transfer unit is implanted into a surrogate mother.

13. A method of producing a cloned non-human mammalian nuclear transfer unit by nuclear transfer according to claim 12 wherein the nuclear transfer unit is becomes an embryo.
